(19) United States
(12) Patent Application Publication
(10) Pub. No.: US 2026/0309591 A1
(43) Pub. Date: Oct. 8, 2026

(54) Personal Health Tracker for Measuring Gas Produced by Gut Microbiota

(71) Applicants: Justin Pratt, San Clemente, CA (US); Sean Costello, Amherst, NY (US); Will Africano, Portland, OR (US)

(72) Inventors: Justin Pratt, San Clemente, CA (US); Sean Costello, Amherst, NY (US); Will Africano, Portland, OR (US)

(21) Appl. No.: 19/180,370

(22) Filed: Apr. 16, 2025

Related U.S. Application Data
(60) Provisional application No. 63/636,105, filed on Apr. 19, 2024.

(51) Int. Cl. G01N 1/22; A61B 5/00; A61B 10/00; E03D 11/13; G16H 10/40; G16H 40/67

(52) U.S. Cl. CPC G01N 1/22; A61B 5/486; A61B 10/00; E03D 11/13; G16H 10/40; G16H 40/67; A61B 2010/0083

(57) ABSTRACT

A system and method for monitoring gastrointestinal health using a passive air-sampling device positioned on top of a toilet tank. The device comprises a gas sensor array, environmental sensors, and a processor configured to collect data during a user's bowel movement. Gas signatures are analyzed using machine learning to identify users and assess gut health without requiring stool handling. A proprietary health score is generated from sensor and contextual data and displayed to the user via a mobile application. The system continuously improves by associating sensor patterns with user-reported symptom classifications and retraining its algorithms to detect gastrointestinal conditions. The neural-network-enabled gas sensor produces multidimensional digital fingerprints of sampled gas compositions, enabling condition screening based on complex patterns rather than predefined biomarkers. The application includes a goal-setting chatbot using cognitive behavioral therapy methods to promote habit change. Data processing is distributed across on-device inference, edge AI, and cloud services.

### FIELD OF THE INVENTION

The present invention relates to personal health monitoring systems. More specifically, it pertains to systems for non-invasive gastrointestinal health assessment using machine learning-based analysis of gas emissions, behavioral context, and interactive user feedback mechanisms.

### BACKGROUND OF THE INVENTION

Monitoring gut health traditionally relies on stool testing, which faces challenges including low compliance and inconvenience. While stool contains valuable diagnostic information, most individuals are reluctant to collect and handle it. As a result, adoption of at-home stool testing remains low despite its potential benefits.

Several systems are known in the art that attempt to monitor toilet emissions for health-related purposes. These include smart toilets that detect volatile compounds from waste, and systems that collect waste samples or image stool in the toilet bowl. However, these systems typically rely on traditional gas threshold sensing, rigid detection rules, or require toilet modification. Furthermore, they generally lack personalized symptom classification, real-time user feedback, and behavior-change interventions.

This invention eliminates the need for direct stool handling and introduces a modular, passive, externally-mounted device capable of detecting complex gas signatures, identifying users, and learning over time to recognize specific gastrointestinal conditions. It further incorporates behavioral therapy principles, AI-assisted goal setting, and dynamic habit coaching through a companion mobile application.

### SUMMARY OF THE INVENTION

This invention is a bathroom-based device placed atop a toilet tank that detects gases released during bowel movements. It uses neural-network-enabled gas sensors, contextual environmental data, and machine learning to assess gastrointestinal health, identify users, and generate a personalized health score viewable via a mobile app. The system incorporates cognitive behavioral therapy (CBT) tools for guided behavior change and personalized health coaching.

Unlike conventional gas sensors that rely on detecting predefined chemical species, the present invention uses sensors capable of generating multidimensional digital fingerprints from complex gas mixtures. This enables the system to learn correlations between volatile compound profiles and symptom states, even without identifying specific biomarker molecules.

The system presents users with standardized clinical surveys (e.g., Rome IV for IBS classification). These responses are used to label gas data collected during bowel events, enabling the machine learning engine to train classification models based on real-world, self-reported symptomology. Over time, this model refines its ability to screen for gastrointestinal conditions such as irritable bowel syndrome (IBS), small intestinal bacterial overgrowth (SIBO), or dysbiosis.

The companion mobile app provides users with health metrics, trend analysis, and a proprietary FloraScore™, derived from longitudinal sensor data. Users may define personal wellness goals via an AI chatbot that incorporates CBT principles, helping to reinforce beneficial habits and reduce negative behaviors.

### BRIEF DESCRIPTION OF THE DRAWINGS

FIG. 1 is a perspective view of the health monitoring device positioned atop a toilet tank in a bathroom setting.

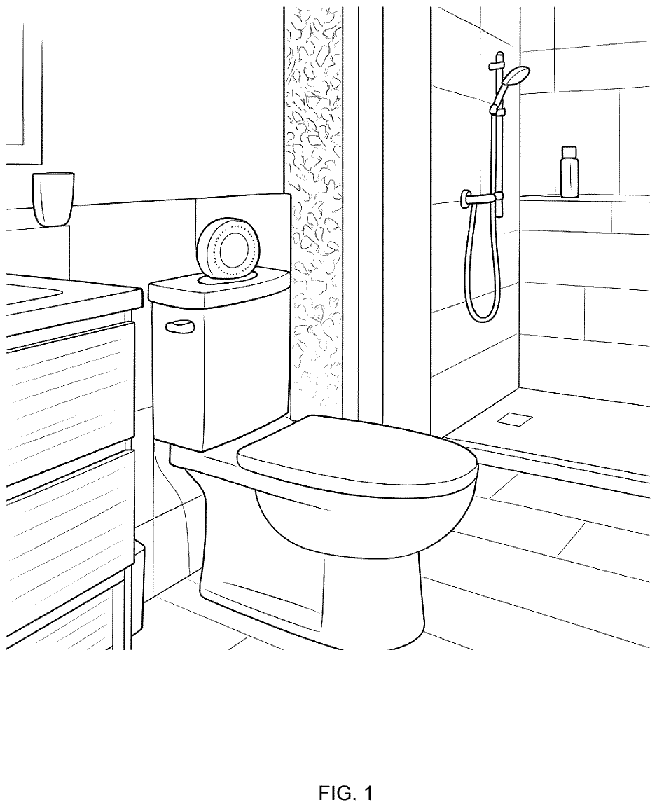

FIG. 2 is an exploded view showing the internal components of the device, including the housing, PCB, system-on-chip, and battery.

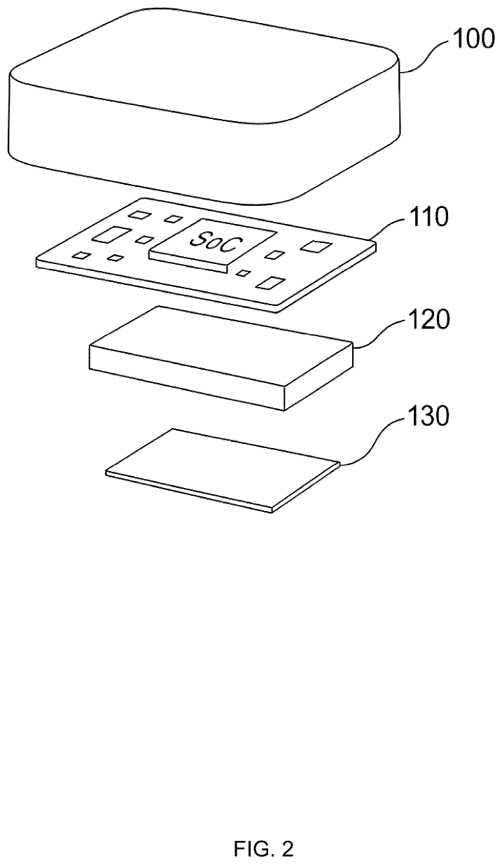

FIG. 3 is a diagram illustrating the data flow from gas detection to user feedback, including ML and LLM layers.

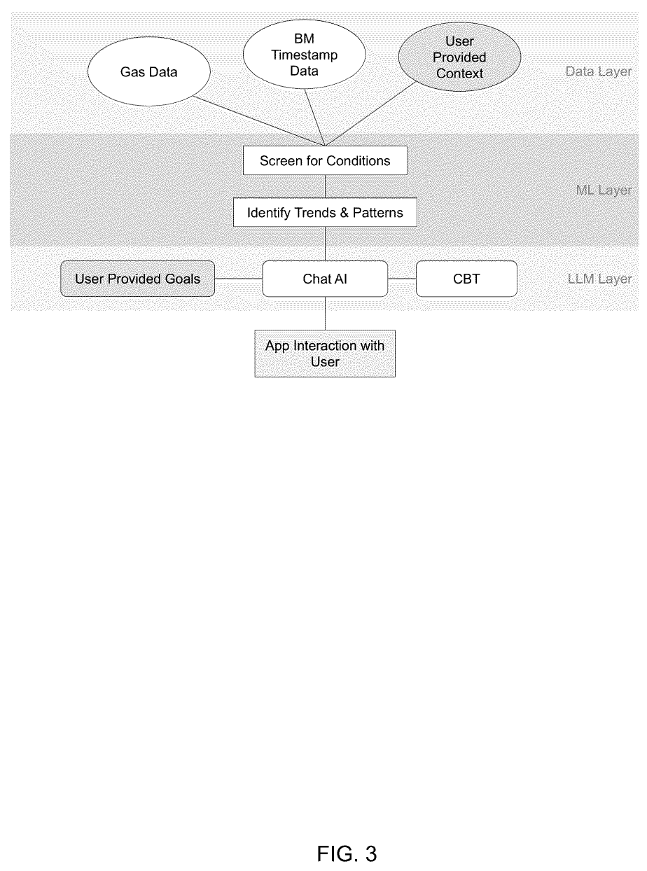

FIG. 4 is a block diagram of the system architecture showing sensor inputs, processing stages, and app output.

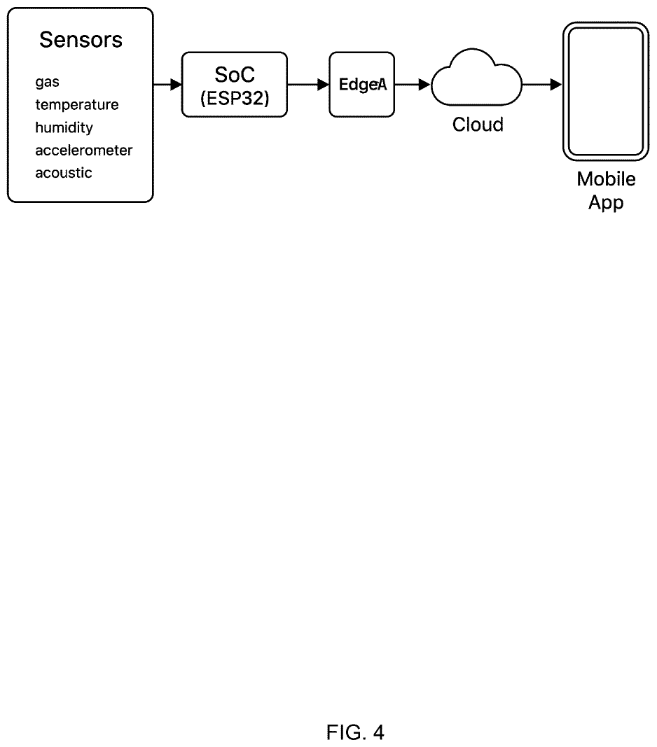

FIG. 5 is a screenshot of the mobile application interface displaying the proprietary FloraScore™ and related health metrics.

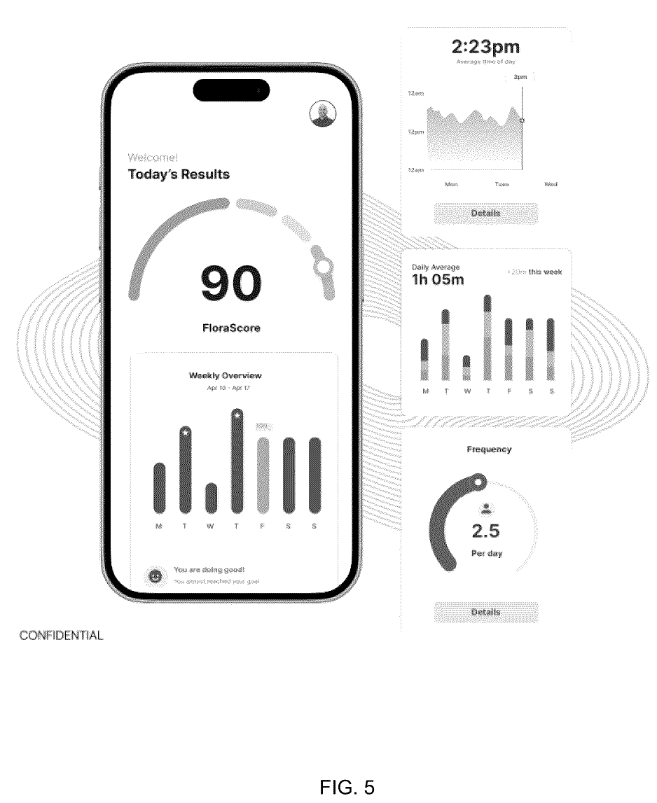

FIG. 6 is a side-view line drawing showing the integration of the device onto a toilet and airflow direction.

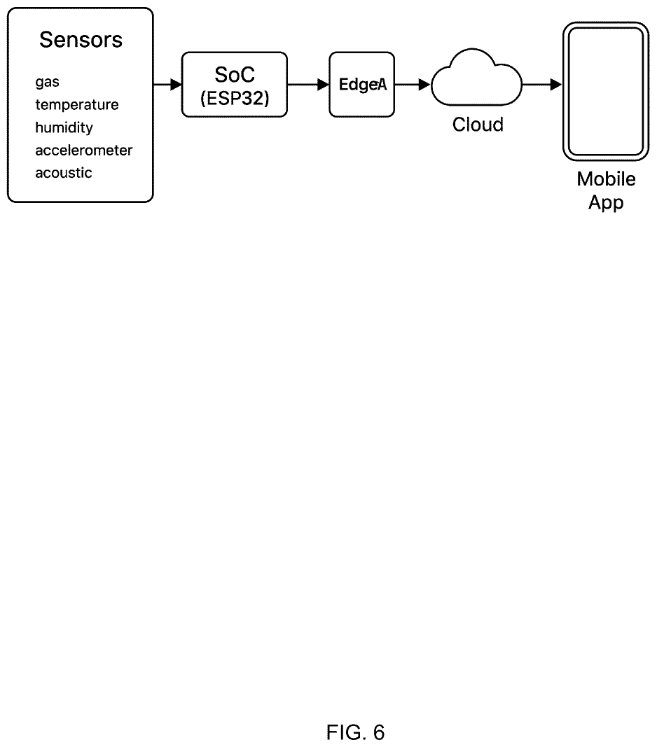

### DETAILED DESCRIPTION OF THE INVENTION

The present invention relates to a personal health monitoring device configured to measure gaseous emissions resulting from gut microbiota metabolism, and to analyze such data for the purpose of gastrointestinal health assessment. The device is intended for use in a residential bathroom setting and is designed to passively collect data during a user's bowel movement, without requiring direct interaction.

The device comprises a housing that rests securely atop the back tank of a standard toilet. Within this housing is an array of gas sensors, including but not limited to: neural-network-enabled metal-oxide semiconductor (MOS) sensors (e.g., Bosch BME688); optional dedicated sensors for hydrogen (H₂), methane (CH₄), and hydrogen sulfide (H₂S); and supporting environmental sensors for temperature, humidity, barometric pressure, acoustic signals, and motion.

Gas sampling is achieved through passive diffusion, eliminating the need for pumps or fans, and minimizing noise and mechanical complexity. The sensor suite enables real-time detection of gases emitted from the stool during defecation, and captures ambient data that supports context-aware signal processing.

The key innovation lies in the use of neural-network-enabled gas sensors, which generate multidimensional digital fingerprints representing the composite gas signature of a bowel movement. These fingerprints are interpreted by a machine learning engine that classifies the event based on pattern recognition, rather than relying on explicit detection of known biomarkers. This enables the system to learn new associations and improve its screening accuracy over time.

The device incorporates a suite of contextual sensors, including a microphone, accelerometer, and proximity modules using Bluetooth and Wi-Fi signals. These inputs are used to infer the presence of a user, identify the individual based on behavioral patterns and environmental context, and reduce false positives. User identification is confirmed during early use and continuously refined using supervised learning.

Data processing is distributed across on-device inference (e.g., TensorFlow Lite on microcontrollers), edge-AI components for more intensive feature extraction, and secure cloud infrastructure for longitudinal analysis, condition screening, and model retraining.

A distinguishing aspect of the invention is its use of user-reported symptom data as training input for the classification engine. The system presents users with standardized surveys (e.g., Rome IV criteria for IBS), and when sufficient correlation is established between symptoms and sensor data, the event is labeled and added to the model's training set. This closed-loop feedback system enables the classifier to evolve as more labeled examples are introduced.

The companion mobile app interfaces with the device to present the user with health trends, the FloraScore™, and event-based insights. The app includes features such as bowel movement tracking, digestive completeness estimation, timing analysis, and compliance logging. It also hosts an AI-powered chat interface where users can define personalized health goals (e.g., reduce bloating, improve frequency) and receive adaptive coaching. The coaching engine employs CBT principles to reinforce habit formation, break undesirable patterns, and encourage sustained behavioral change.

All data is handled in compliance with relevant health privacy laws (e.g., HIPAA, GDPR). User participation in labeling, goal setting, or training is opt-in and privacy-controlled.

1. A personal health monitoring system for gastrointestinal analysis, comprising:
a housing configured to rest on top of a toilet tank;
one or more gas sensors, including a neural-network-enabled metal-oxide semiconductor (MOS) sensor, disposed within the housing and configured to generate digital gas fingerprints from emissions released during a user's bowel movement;
at least one environmental or behavioral sensor selected from the group consisting of temperature, humidity, barometric pressure, acoustic, motion, and wireless proximity sensors;
a passive diffusion intake pathway configured to allow ambient gas entry without active airflow components;
a processor comprising a system-on-chip (SoC), configured to preprocess sensor data and communicate wirelessly with external devices;
a machine learning engine trained to identify individual users based on multi-modal contextual data;
a mobile application configured to:
receive and display personalized gastrointestinal health metrics;
present a proprietary gut health score derived from the sensor data; and
provide behavior change support via an AI chatbot that incorporates cognitive behavioral therapy techniques.

2. A method for assessing gastrointestinal health using a passive toilet-mounted device, the method comprising:
passively collecting air samples containing gases released during a bowel movement via a gas sensor array positioned atop a toilet tank;
generating a multidimensional digital gas fingerprint using a neural-network-enabled gas sensor;
collecting contextual data including time, temperature, humidity, acoustic profile, motion, and wireless proximity;
associating collected data with a specific user using a trained identification model;
receiving symptom classification data from the user via a structured diagnostic survey aligned with a standardized clinical framework;
labeling bowel movement data with the corresponding symptom classification to create a supervised training dataset;
training or refining a machine learning model to detect gastrointestinal symptom patterns based on labeled gas fingerprint data;
generating a proprietary gut health score based on gas and contextual signals;
presenting the gut health score and trend visualizations in a user-facing mobile application; and
generating behavioral guidance using a chatbot trained in cognitive behavioral therapy methods to help the user achieve health goals.

3. The system of claim 1, wherein the neural-network-enabled MOS sensor is configured to: classify bowel emissions based on gas signal fingerprints without isolating predefined chemical biomarkers.

4. The system of claim 1, wherein the housing contains an integrated battery and electronics module and is designed for user-removable installation on a toilet tank.

5. The system of claim 1, wherein the wireless communication comprises both Bluetooth and Wi-Fi protocols.

6. The method of claim 2, wherein the structured diagnostic survey corresponds to Rome IV criteria for irritable bowel syndrome.

7. The method of claim 2, wherein the symptom classification is used to generate a continuously updating, user-specific classifier capable of screening for gastrointestinal conditions.

8. The mobile application of claim 1, wherein the chatbot provides adaptive recommendations based on detected bowel trends and uses CBT reinforcement strategies.

9. The system of claim 1, wherein data processing is distributed across on-device microcontroller inference, edge processing hardware, and secure cloud servers.

10. The method of claim 2, wherein bowel movement completeness, frequency, and timing intervals are used as features in the health score computation.
